(19) United States
(12) Patent Application Publication
(10) Pub. No.: US 2006/0235499 A1
(43) Pub. Date: Oct. 19, 2006

(54) Coated lead fixation electrode

(75) Inventors: Ronald W. Heil JR., Roseville, MN (US); Michael T. Nelson, White Bear Lake, MN (US)

Correspondence Address: GUIDANT PATENT DOCKETING;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/105,791

(22) Filed: Apr. 14, 2005

(51) Int. Cl. A61N 1/05

(52) U.S. Cl. 607/127

(57) ABSTRACT

In one embodiment, the present invention provides a cardiac lead including a fixation member extending from a distal end of the lead. The fixation member includes a coated portion and an uncoated portion. The coating that forms the coated portion includes a biocompatible polymer material and a therapeutic agent. The coating may provide for the delivery of the therapeutic agent directly at the sight of fixation for an extended period of time.

### TECHNICAL FIELD

The present invention relates to cardiac rhythm management systems, and in particular to mechanisms for improving the performance of cardiac leads implanted in a patient's vascular system.

### BACKGROUND

When functioning properly, the human heart maintains its own intrinsic rhythm, and is capable of pumping adequate blood throughout the body's circulatory system. However, some people have irregular cardiac rhythms, referred to as cardiac arrhythmias. Such arrhythmias result in diminished blood circulation. One manner of treating cardiac arrhythmias includes the use of a cardiac rhythm management system. Such systems may be implanted in a patient to deliver electrical pulses to the heart.

Cardiac rhythm management systems include, for example, pacemakers (also referred to as “pacers”), defibrillators (also referred to as “cardioverters”) and cardiac resynchronization therapy (“CRT”) devices. These systems use conductive leads having one or more electrodes to deliver pulsing energy to the heart, and may be delivered to an endocardial, epicardial and myocardial position within the heart.

Unfortunately, interactions between the electrode and the adjacent tissue in the heart may vary the stimulation thresholds of the tissue over time. This variation may be caused by the formation of fibrotic scar tissue during the recovery and healing process as the body reacts to the presence of the electrode. The formation of fibrotic tissue may result in chronic stimulation energy thresholds that exceed the acute energy thresholds obtained immediately after implant. As a result, higher stimulation energies are required, thereby shortening the usable life of the battery-powered implantable cardiac rhythm management device.

There is a need in the art for cardiac leads that deliver therapeutic agents such as steroids directly to the site at which the electrode is positioned in order to maintain consistent stimulation energy thresholds throughout the life of the lead.

### SUMMARY OF THE INVENTION

In one embodiment, the present invention provides a lead for use in a cardiac rhythm management system. The lead includes a lead body having a proximal end, a distal end, and a conductive member extending between the proximal and distal ends. The lead further includes a fixation electrode coupled to the lead body, which is in electrical communication with the conductive member. A biocompatible coating including a polymeric material and a therapeutic agent is applied over a portion of the fixation electrode.

Suitable therapeutic agents include steroids, and in particular, esters of steroids. Suitable polymeric materials may generally resist degradation in vivo, and include medical grade silicone polymers. The combination of the therapeutic agent and polymeric material may be selected to provide immediate and/or extended treatment in vivo.

The coating may be applied to the fixation electrode in order to provide the electrode with discrete coated and exposed portions. For example, the distal end of the electrode may remain exposed, while the proximal end is coated. In another example, alternating exposed and coated portions may be utilized. In a further example, the coating is applied onto a polymer sleeve disposed over a portion of the electrode. Suitable polymer sleeves may act as a substrate to receive the coating and/or as an insulator over portions of the fixation electrode.

In another embodiment, the present invention provides a cardiac rhythm management system including a pulse generator, a lead having an electrode and a proximal end coupled to the pulse generator, and a coating means disposed over a portion of the electrode for providing an extended release of an anti-inflammatory therapeutic agent.

In yet another embodiment, the present invention provides a method for coating an electrode on a cardiac lead. A coating mixture including a polymeric material and a therapeutic agent applied onto the electrode such that the electrode includes a coated and an uncoated portion. The applied coating mixture may then be treated, for example by drying and/or curing, to form a coating on the electrode.

The coating may be applied in several ways. In one embodiment, the coating is brushed, sprayed or otherwise applied onto a portion of the electrode to provide a coated and an exposed portion. In another embodiment, the coating is applied onto a polymer sleeve disposed over a portion of the electrode. Optionally, a portion of the polymer sleeve may then be removed to provide the electrode with a coated and an exposed portion. In yet another embodiment, a portion of the electrode is masked prior to application of the coating mixture. After application of the coating mixture, the masked portions of the electrode are de-masked to provide the electrode with a coated and an exposed portion.

In a further embodiment, the present invention provides a coating for application to an electrode on a cardiac lead. The coating includes one or more of the polymeric materials and therapeutic agents reported herein.

In yet a further embodiment, the present invention provides a coating for a medical device, which includes a mixture of a polymeric material and a lipophilic ester of a steroid, for example the acetate ester of dexamethasone.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic illustration of distal portions of the cardiac rhythm management system implanted in a patient's heart according to one embodiment of the present invention.

FIG. 2 is a schematic illustration of the distal end of a cardiac lead according to one embodiment of the present invention.

FIG. 3 is a schematic illustration of the distal end of a cardiac lead according to one embodiment of the present invention.

FIG. 4 is a schematic illustration of the distal end of a cardiac lead according to one embodiment of the present invention.

FIG. 5 is a schematic illustration of the distal end of a cardiac lead according to one embodiment of the present invention.

FIG. 6 is a flow-chart summarizing a method of using embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

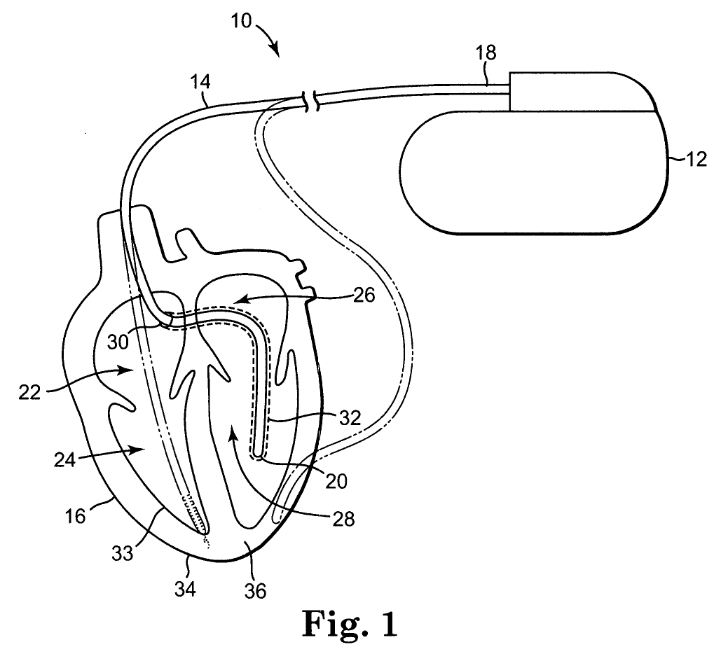

FIG. 1 is a schematic drawing of a cardiac rhythm management system 10 including an implantable cardiac rhythm management device 12 and a lead 14, which electrically couples the cardiac rhythm management device 12 to a patient's heart 16. The lead 14 includes a proximal end 18 attached to the cardiac rhythm management device 12 and a distal end 20 which is implanted in the patient's heart 16.

FIG. 1 further illustrates the chambers of the heart 16, including a right atrium 22, a right ventricle 24, a left atrium 26, and a left ventricle 28. In the embodiment illustrated in FIG. 1, the lead 14 is an endocardial lead, and the distal end 20 of the lead 14 extends transvenously through the right atrium 22, through a coronary sinus 30, and into a great cardiac vein 32. The illustrated disposition of the lead 14 may be used for delivering pacing and/or defibrillation energy to the left side of the heart 16, for the treatment of congestive heart failure (CHF) or other cardiac disorders requiring therapy delivered to the left side of heart 16.

FIG. 1 shows in phantom an alternate endocardial disposition of the lead 14, which extends through the right atrium 22 and/or the right ventricle 24 and is directly implanted in the endocardium 33. As further shown in phantom in FIG. 1, the lead 14 may also be directly implanted in an epicardial 34 or myocardial 36 position in the heart 16. Epicardial and myocardial lead disposition is generally accomplished by piercing a patient's chest wall and affixing the lead 14 directly into the epicardium 34 or myocardium 36.

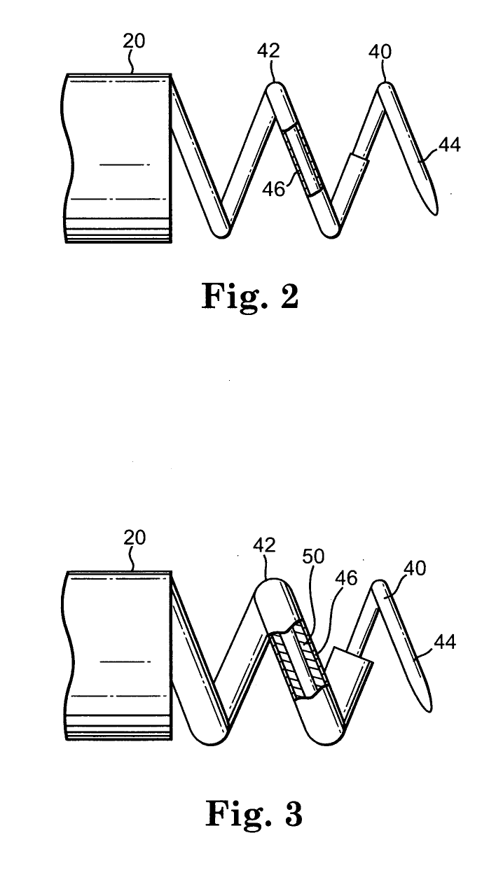

FIG. 2 shows an enlarged view of the distal end 20 of the lead 14. A fixation electrode 40 is coupled to the distal end 20 of the lead 14 such that the fixation electrode 40 is in electrical communication with the cardiac rhythm management device 12 (See FIG. 1). The fixation electrode 40 includes a coated proximal portion 42 and an exposed distal portion 44. The cut-away portion of FIG. 2 is for illustrative purposes and indicates that the coated portion 42 comprises a thin coating 46.

The exposed portion 44 of the fixation electrode 40 is conductive and is designed to be positively fixed to the desired site in the heart 16 to deliver current to the heart for cardiac rhythm management therapy. The coating 46 that forms the coated portion 42 is designed to release one or more therapeutic agents directly at the fixation site.

FIG. 3 shows an alternate embodiment of the present invention in which the coated portion 42 includes a polymer sleeve 50 positioned over a portion of the fixation electrode 40. The coating 46 is applied onto the polymer sleeve 50. One benefit to this configuration is that the polymer sleeve 50 may provide an improved substrate onto which the coating 46 may be applied, and/or that the polymer sleeve 50 may also serve as an insulator with respect to the fixation electrode 40. The polymer sleeve 50 may be formed from silicone rubber or another biocompatible polymer or mixture of polymers. In one embodiment, the sleeve has a thickness of approximately 0.004 in.

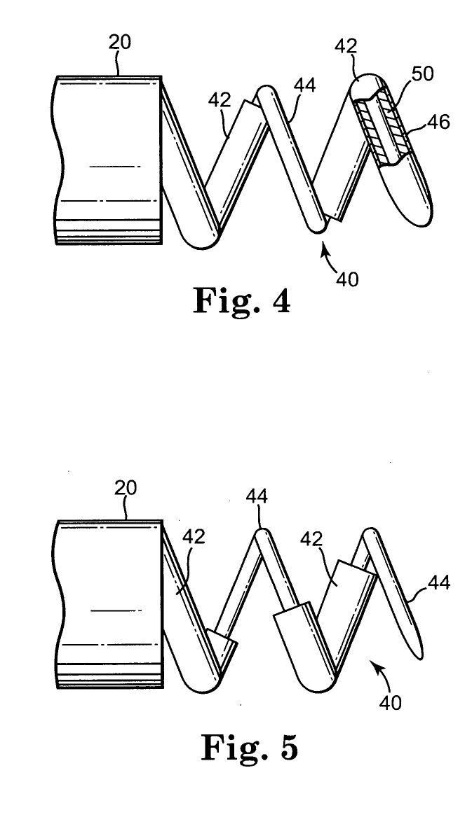

FIG. 4 shows another embodiment of the present invention in which two coated portions 42 are positioned over discrete proximal and distal portions of the fixation electrode 40. The exposed portion 44 is disposed in between the coated portions 42. FIG. 5 shows a further embodiment of the present invention in which the fixation electrode 40 includes alternating segments of coated portions 42 and exposed portions 44 to provide several discrete conductive regions.

Although FIGS. 2-5 illustrate the fixation electrode as generally having a coiled or helical shape, a wide variety of conventional shapes and sizes may also be used for the fixation electrode 40. Additionally, the relative length of the coated and uncoated portions 42, 44 shown in FIGS. 3-6, may vary depending on the particular application. Thus, in certain embodiments coated portion 42 may make up a majority of the fixation electrode 40. In other embodiments, the exposed portion 44 may form the majority of the fixation electrode 40. In yet another embodiment, the coating 46 could be applied to a conventional non-fixation electrode, such as a ring electrode, to provide coated and uncoated portions 42, 44. In this embodiment, discrete fixation means such as tines, sutures or other conventional fixation devices could be used to fix the lead at a desired position.

The coating 46 generally includes a biocompatible polymer material and a therapeutic agent. A wide range of biocompatible polymer materials capable of carrying and delivering a therapeutic agent may be utilized in embodiments of the present invention. In one embodiment, the polymer material may remain coated to the fixation electrode 40 for the life of the lead 14. In another embodiment, the polymer material may be partially or completely biodegradable over time. In a further embodiment, the polymer material may have a low water solubility or may be substantially water insoluble (collectively referred to herein as “low water solubility”). In alternative embodiments, the polymer material may swell when contacted with water or other aqueous mixtures. Suitable polymeric materials should also be compatible with the therapeutic agent with which the polymeric material is combined

Examples of suitable polymer materials include silicone rubbers, polyurethanes, polyesters, polylactic acids, polyamino acids, polyvinyl alcohols and polyethylenes. Medical grade silicone rubbers may be particularly suitable for embodiments of the present invention.

Suitable therapeutic agents for incorporation into the coating 46 may treat the tissue surrounding the fixation site of the fixation electrode 40, for example by providing an anti-inflammatory effect, in order to maintain or reduce the chronic energy thresholds required to provide rhythm management therapy to the heart 16. Steroids are a broad class of therapeutic agents that may be suitable for use in embodiments of the present invention. Examples of suitable steroids include dexamethasone, betamethasone, paramethasone, beclomethasone, clobetasol, triamcinolone, prednisone, and prednisolone, as well as combinations and derivatives thereof. Suitable steroid derivatives include esters of steroids, such as the acetate, diacetate, propionate, dipropionate, cypropionate, butyrate, acetonide, hexacetonide, succinate and valerate esters of such steroids. The acetate ester of dexamethasone or beclomethasone may be particularly suitable for certain embodiments. Beclomethasone dipropionate anhydrous may also be suitable for certain embodiments. A separate class of therapeutic agents that may be suitable for certain embodiments include anti-cell proliferation agents such as paclitaxel (sold as Taxol® by Bristol-Myers Squibb) and Docetaxel® (Rhone-Poulenc Rorer).

The specific combination of polymer and therapeutic agent, and the relative concentrations of these materials, may vary depending on the type of lead implanted, the location of lead implantation, and the anticipated length of time that the lead is to remain implanted. In embodiments in which extended treatment with a therapeutic agent is desired, It may be desirable to utilize a combination of a polymer and therapeutic agent that provides an extended release of therapeutic agent. In one embodiment, the polymer and therapeutic agent may be selected such that the therapeutic agent generally blends well with, and/or is substantially soluble in, the polymeric material and/or any solvent in which the therapeutic agent and polymeric material are combined prior to application to the electrode. For example, dexamethasone acetate may be combined with silicone rubber to provide both an immediate release of therapeutic agent from the surface of the coating and an extended diffusion of therapeutic agent from the remainder of the coating. This treatment may be further enhanced because the agent is released from the coating at the point of fixation.

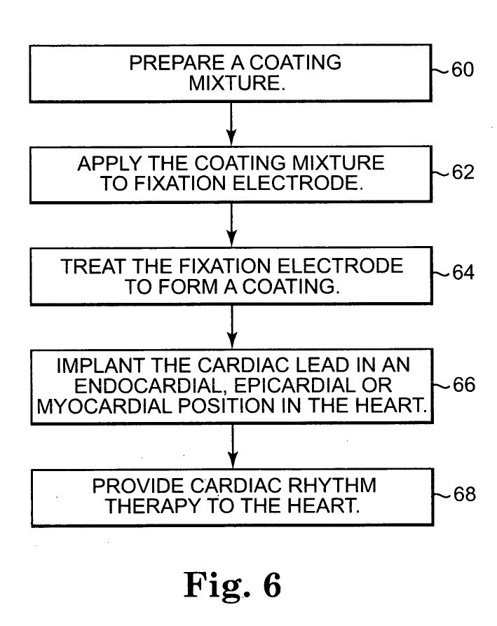

FIG. 6 shows a flow-chart summarizing a method of preparing and using embodiments of the present invention. First, a coating mixture is prepared for application to the fixation electrode (block 60).

In one embodiment, the coating mixture includes a combination of uncured or cured polymer material, one or more therapeutic agents, and an organic or substantially organic carrier liquid. As used herein, the term “coating mixture” encompasses solutions, dispersions, emulsions and other mixtures of solid materials with one or more liquids. Examples of suitable carriers for use in the coating mixture may include or contain Freon, hexane, heptane and/or xylene. In another embodiment, certain uncured polymers and therapeutic agents may be combined free of solvent.

Generally speaking, the coating mixture may be applied as a thin coating 46 to the fixation electrode. 40 or the polymer sleeve 50 by conventional methods, including dip, brush and spray application (block 62). After applying of the coating mixture, the solvent is dried or dispersed by heat or air-drying to form a thin coating 46 (block 64). Optionally, the coating 46 may then be partially or fully cured via known methods under conditions that do not adversely affect the potency of the therapeutic agent. The resulting coating 46 may have a thickness of between about 1 and about 100 microns, more particularly between about 10 and about 80 microns, and even more particularly between about 13 and about 76 microns. The thickness of the coating may be increased by applying multiple layers of the coating mixture. Such multiple coatings do not necessarily need to utilize the same combination of polymer and therapeutic agent in each layer. Instead, different polymers and/or therapeutic agents could be utilized in discrete layers to provide a desired therapeutic affect.

In one embodiment, the polymer sleeve 50 is first positioned over a proximal portion of the electrode 40. The placement of the polymer sleeve 50 may be accomplished by swelling the sleeve with a suitable solvent, placing the polymer sleeve 50 over the electrode 40, and then drying the polymer sleeve 50. The coating mixture is then applied onto the polymer sleeve 50 by brush application for example, so that the proximal portion of the electrode 40 is coated while the distal end of the electrode 40 remains uncoated and exposed.

In an alternate embodiment, the polymer sleeve 50 is positioned over most or all of the electrode 40. The entire polymer sleeve 50 is then coated with the coating mixture by dip coating for example. After the coating 46 has formed, a portion of the resulting coating 46 and polymer sleeve 50 may be cut, stripped, dissolved or otherwise removed to expose a desired portion of the electrode 40.

In yet another embodiment, a portion of the electrode 40 is covered with a conventional masking material. The coating mixture may then be applied to the entire electrode 40 (with or without a polymer sleeve 50 positioned over a portion of the electrode) by dip application or another suitable application method. After the coating 46 has formed, the masking agent may be removed to reveal an exposed portion of the electrode 40.

In certain embodiments such as those illustrated in FIGS. 4 and 5, it may be desirable to leave exposed a portion of the fixation electrode 40 other than the distal portion 26. In such embodiments, the entire electrode 40 may first be covered with the polymer sleeve 50. The coating mixture is then applied to the entire polymer sleeve 50 via dip application or another suitable application method. After curing, the desired portion of the polymer sleeve 50 and the coating 46 is cut, stripped or otherwise removed from the fixation electrode 40 to reveal an exposed portion. A fixation electrode 40 having alternating exposed and coated portions may be formed in a similar manner.

As further shown in FIG. 6, after coating, the lead 14 may be implanted, as described with respect to FIG. 1, in a patient's heart 16 (block 66). Specifically, the lead 14 of the present invention may be suitable for implantation in the patient's epicardium, endocardium or myocardium. Pacing therapy may then be applied to the patient's heart in a conventional manner (block 68).

Various modifications and additions can be made to the exemplary embodiments discussed without departing from the scope of the present invention. Accordingly, the scope of the present invention is intended to embrace all such alternatives, modifications, and variations as fall within the scope of the claims, together with all equivalents thereof.

We claim:

1. A lead for use in a cardiac rhythm management system, the lead comprising:
a lead body including a proximal end and a distal end and a conductive member extending between the proximal and distal ends;
a fixation electrode coupled to the lead body and in electrical communication with the conductive member; and
a biocompatible coating applied over a portion of the fixation electrode, the coating including a polymeric material and a therapeutic agent.

2. The lead of claim 1 wherein the fixation electrode includes a coated portion and an exposed portion.

3. The lead of claim 2 wherein the coated portion covers a proximal portion of the fixation electrode.

4. The lead of claim 2 comprising a plurality of alternating coated and exposed portions.

5. The lead of claim 1 wherein the fixation electrode comprises a positive fixation electrode.

6. The lead of claim 1 wherein the fixation electrode comprises a helical or coiled portion.

7. The lead of claim 1 further comprising a polymer sleeve disposed over a portion of the fixation electrode, and wherein the coating is applied onto a portion of the polymer sleeve.

8. The lead of claim 1 wherein the polymeric material is substantially insoluble in water.

9. The lead of claim 1 wherein the polymeric material comprises silicone.

10. The lead of claim 1 wherein the therapeutic agent comprises a steroid.

11. The lead of claim 10 wherein the steroid is selected from the group consisting of dexamethasone, betamethasone, paramethasone, beclomethasone, clobetasol, triamcinolone, prednisone, prednisolone and combinations and derivatives thereof.

12. The lead of claim 10 wherein the therapeutic agent comprises an ester of a steroid.

13. The lead of claim 10 wherein the therapeutic agent comprises an acetate, diacetate, propionate, dipropionate, cypropionate, butyrate, acetonide, hexacetonide, succinate or valerate ester of a steroid.

14. The lead of claim 13 wherein the therapeutic agent comprises an acetate ester of dexamethasone and the polymeric material comprises silicone.

15. The lead of claim 1 wherein the coating includes a first layer comprising a first coating mixture and a second layer comprising a second coating mixture.

16. A cardiac rhythm management system comprising
a pulse generator;
a lead having an electrode and a proximal end adapted for coupling with the pulse generator;
a coating means for providing an extended release of a anti-inflammatory therapeutic agent, the coating means being disposed over a portion of the electrode; and
means for affixing the lead in a patient.

17. A method of coating a cardiac lead electrode comprising:
providing a coating mixture including a polymeric material and a therapeutic agent;
applying the coating mixture onto the electrode such that the electrode includes a coated portion and an exposed portion; and
treating the coating mixture to form a coating on the electrode.

18. The method of claim 17 wherein the coating mixture is applied to the electrode by spraying, brushing or dipping.

19. The method of claim 17 wherein the electrode includes a polymer sleeve, and the coating mixture is applied onto the polymer sleeve.

20. The method of claim 19 wherein a portion of the polymer sleeve is removed after applying the coating mixture to provide the coated and exposed portions.

21. The method of claim 17 further comprising the steps of masking a portion of the electrode prior to applying the coating mixture, and de-masking the portion of the electrode after applying the coating mixture to provide the exposed portion.

22. The method of claim 17 wherein the treating step comprises drying the coating mixture, curing the coating mixture, or both.
